(19) United States
(12) Patent Application Publication
(10) Pub. No.: US 2008/0193937 A1
(43) Pub. Date: Aug. 14, 2008

(54) Identification of a Sterol Acyltransferase Gene

(76) Inventors: Jitao Zou, Saskatchewan (CA); Qilin Chen, Saskatchewan (CA)

Correspondence Address: ADE & COMPANY INC., 2157 Henderson Highway, WINNIPEG, MB, R2G1P9, omitted

(21) Appl. No.: 11/910,065

(22) PCT Filed: Mar. 30, 2006
(86) PCT No.: PCT/CA2006/000476

§ 371 Date: Dec. 3, 2007

Related U.S. Application Data
(60) Provisional application No. 60/666,520, filed on Mar. 30, 2005.

(51) Int. Cl. C12Q 1/68; C12P 21/04

(52) U.S. Cl. 435/6; 435/69.1

(57) ABSTRACT

The present invention relates to the use of genetic engineering to produce sterol esters. In one embodiment, an isolated or recombinant nucleic acid molecule encoding a sterol acyltransferase is disclosed. In another embodiment, a cell transformed with the isolated or recombinant nucleic acid molecule encoding a sterol acyltransferase is disclosed. A process for producing sterol esters using the transformed cell is also disclosed. In a further embodiment; an isolated or recombinant sterol acyltransferase is disclosed.

### PRIOR APPLICATION INFORMATION

This application claims the benefit of U.S. Provisional Application 60/666,250, filed Mar. 30, 2005.

### TECHNICAL FIELD

The present invention relates generally to biotechnology, and more particularly, to the use of genetic engineering to produce sterol esters.

### BACKGROUND OF THE INVENTION

The ability of phytosterols to lower low density lipoprotein (“LDL”) cholesterol in human subjects as part of a diet has been established in the medical field. Phytosterol-containing foods having therapeutic value have been approved for use by the Food and Drug Administration (“FDA”) and are available on the market.

However, free phytosterols are difficult to incorporate into food stuffs due to the low solubility of the free phytosterols. Phytosterol esters can, on the other hand, be dissolved in oil at a concentration ten times higher than that of the free phytosterols. Thus, the current commercial production of phytosterol-containing foods requires a costly fatty acid acylation procedure.

Further, since phytosterol esterification processes in planta may represent one biochemical bottleneck that limits phytosterol biosynthesis and, hence, the amount of phytosterols produced, there exists a need for a more efficient process for the production of sterol-esters.

### SUMMARY OF THE INVENTION

In one embodiment, a sterol acyltransferase gene is identified. The sterol acyltransferase gene may be expressed or overexpressed in a cell and used to enhance sterol-ester production in the cell. In another embodiment, the sterol acyltransferase gene is expressed or overexpressed in planta in order to enhance the production of sterol-esters in a crop. In an additional embodiment, a plant, plant seed or progeny thereof includes the sterol acyltransferase gene.

In another embodiment, the sterol acyltransferase gene may be expressed or overexpressed in a cell and used to enhance the biosynthesis and accumulation of sterols in the cell. In as additional embodiment, the sterol acyltransferase gene is expressed or overexpressed in planta in order to increase the total content of sterols in a crop. In yet a further embodiment, a plant, plant seed or progeny thereof includes the sterol acyltransferase gene.

In another embodiment, a vector having a sterol acyltransferase gene is disclosed. The vector may be used to transform a cell, thus producing a recombinant cell having the sterol acyltransferase gene. The cell may comprise a bacterial cell, a yeast cell or a plant cell. In another embodiment, the cell expresses the sterol acyltransferase gene and produced a sterol acyltransferase peptide that may be isolated or purified from the cell. The isolated or purified sterol acyltransferase peptide may be used to generate antibodies having utility in diagnostics or further studies.

In yet a further embodiment, the nucleotide and deduced amino acid sequence associated with a sterol acyltransferase gene are disclosed. The sequence, or a portion of which, may be used to identify genes from other species that encodes a polypeptide with sterol acyltransferase activity. The nucleotide sequence may be used to transform a cell, thus producing a recombinant cell having the sterol acyltransferase gene. The cell may comprise a bacterial cell, a yeast cell or a plant cell.

In one embodiment, a process for producing sterol esters includes transforming a cell with a sterol acyltransferase gene. The transformed cell expresses the sterol acyltransferase gene and produces sterol esters. The sterol esters may be isolated or purified from the recombinant cell or culture media in which the cell grows, and subsequently incorporated into a composition as described herein.

In yet an additional embodiment, the sterol-esters produced with the process of the instant invention are administered in combination with the active ingredients of pharmaceutical or nutraceutical compositions such as, for example, cholesterol lowering agents. Non-limiting examples of cholesterol lowering agents include, without limitation, plant sterols, psyllium, beta glucan, niacin, guggul extract, red rice yeast extract, policosanol, garlic, fenugreek, rice bran oil, fish oil, flaxseed oil, borage oil, other omega-3-fatty acid containing oils, and combinations of any thereof.

In an additional exemplary embodiment, the sterol-esters produced by the processes of the instant invention are incorporated in a food product, such as a beverage or a food, a multi-ingredient nutritional supplement or any combination thereof. Non-limiting examples of food products that the sterol-esters may be incorporated into include cholesterol lowering margarine, soy protein, nuts, flaxseed, olive oil, fish oil, any other oil, and combinations of any thereof.

The sterol-esters produced by the process of the instant invention can be used directly as food additives or admixed with a consumable carrier to a used as the food additive or food composition. One food additive of the invention includes the sterol-esters and a consumable carrier.

In another embodiment, an article of manufacture includes a container for holding an amount of a composition comprising sterol-esters of the instant invention, and indicia associated with the container. The indicia are organized to guide a reader of the indicia to ingest an effective amount of the composition sufficient to help lower or reduce the cholesterol content of a subject that ingests the composition.

It is also contemplated that sterol-esters produced by the process of the instant invention may be prepared in capsule, tablet or liquid form for regular administration to help treat conditions involving high cholesterol.

In another embodiment, the sterol-esters produced by the process of the instant invention are administered as a pharmaceutical or nutraceutical composition to a subject. Such a composition includes the sterol-esters and a pharmaceutically acceptable carrier such as, for example, lactose, cellulose, or equivalent, or contained within a pharmaceutical dosage such as a capsule or tablet and may be used in combination with other pharmaceutical or nutraceutical active ingredients, or cosmetic ingredients that improve the appearance (a. e., color) of the sterol-esters.

According to a first aspect of the invention, there is provided an isolated or recombinant nucleic acid molecule encoding a plant sterol acyltransferase having at least 70% homology to SEQ ID No. 2.

According to a second aspect of the invention, there is provided a cell transformed with the isolated or recombinant nucleic acid molecule as described above.

According to a third aspect of the invention, there is provided a process for increasing sterol ester production in a cell, the process comprising:

transforming a cell with a nucleic acid molecule encoding a sterol acyltransferase; and

growing the cell under conditions wherein said sterol acyltransferase is expressed.

According to a fourth aspect of the invention, there is provided an isolated plant sterol acyltransferase, comprising an amino acid sequence that is at least 70% homologous to SEQ ID NO: 3.

According to a fifth aspect of the invention, there is provided a plant haying a genomic knock-out of a sterol acyltransferase gene.

According to a sixth aspect of the invention, there is provided a method of identifying sterol acyltransferase genes comprising:
(a) transforming a yeast mutant deficient in sterol ester synthesis with a nucleic acid molecule comprising a plant cDNA suspected of encoding a sterol acyltransferase; and
(b) detecting sterol ester formation in said yeast, wherein presence of sterol esters indicates that said plant cDNA encodes a functional sterol acyltransferase.

### BRIEF DESCRIPTION OF THE DRAWINGS

FIG. 1 is a genetic map of pYES2.1N5-His-TOPO®.

FIG. 2 is the nucleotide sequence of At3 g51970 (SEQ NO: 1).

FIG. 3 is the nucleotide sequence of the CDS of the plant sterol acyltransferase gene of Arabidopsis (SEQ ID NO: 2).

FIG. 4 is the predicted amino acid sequence of At3g51970 (SEQ ID NO: 3).

FIG. 5 illustrates HPLC chromatograms demonstrating the production of sterol-esters in cells transformed with At3g51970.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

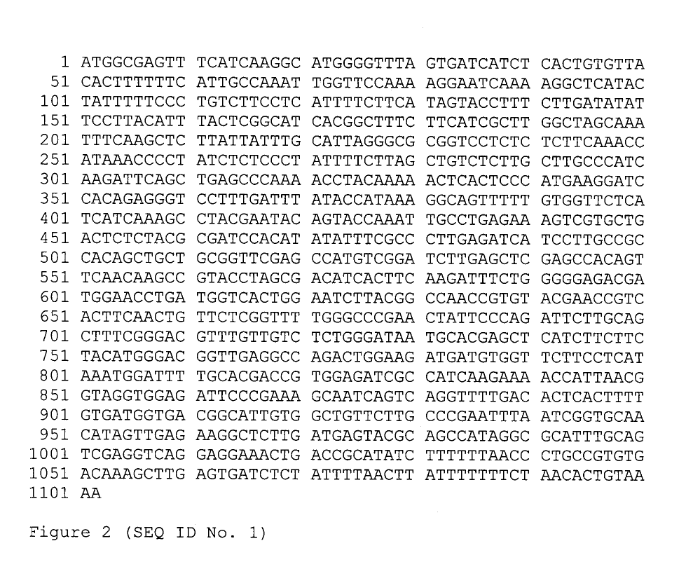

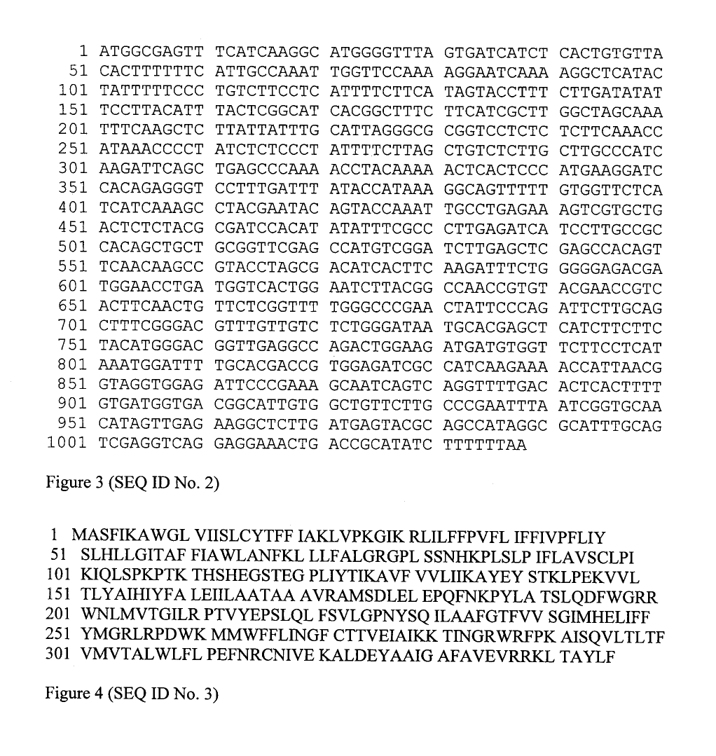

In one embodiment, a sterol acyltransferase gene is identified with a yeast complementation approach in combination with high performance liquid chromatography (HPLC) analysis of neutral lipids in a yeast extract. Specifically, the genomic sequence of the sterol acyltransferase gene is shown in FIG. 2 (SEQ ID No. 1), the coding sequence of the acyltransferase gene is shown in FIG. 3 (SEQ ID No. 2) and the amino acid sequence of the acyltransferase enzyme is shown in FIG. 4 (SEQ ID No. 3).

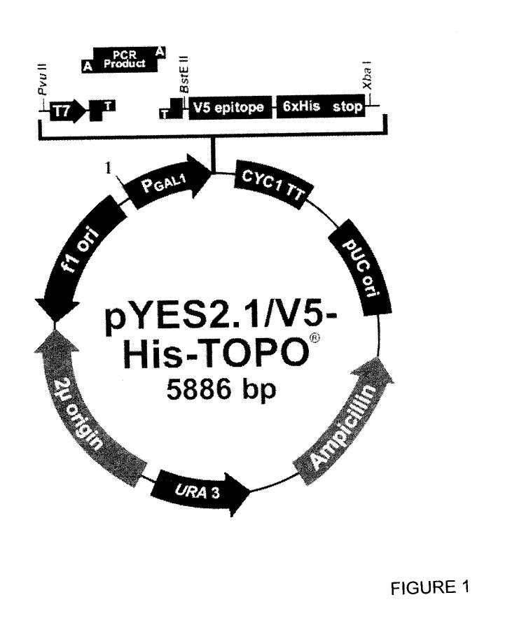

A class of Membrane Binding O-Acyltransferase motif-containing genes may be found in Arabidopsis based on interrogation of a genomic database, combined with an assumption that phytosterol acyltransferase is a membrane-bound acyltransferase. Since not all of the cDNAs are expected to encode a functional sterol acyltransferase, a biochemical functional characterization of the gene products is performed in a yeast strain defective in sterol ester production to discover the sterol acyltransferase gene. The members of cDNAs of the genes found with the interrogation are obtained by RT-PCR using seedling and/or silique RNA as a template. The cDNAs are cloned into a yeast expression vector such as, for example, the readily and commercially available plasmid pYES2.1N5-His-TOPO® (FIG. 1), and the plasmids having the cDNAs are introduced into a yeast mutant strain such as for example, are1are2 that is deficient in sterol ester synthesis.

For selection, the transformed yeast is cultured in dropout SC medium that is -his-leu-ura at 28° C. for 2 days. The yeast expression vector carries a URA gene and the double mutant yeast itself is able to synthesize histidine and leucine. The yeast transformants, upon being induced by galactose, are subjected to normal-phase HPLC analysis of neutral lipid extracts to detect the distinct peak that corresponds to the sterol ester. If a peak appears at the very retention time of sterol ester, the gene whose products possess a function of acylating sterol is identified to produce sterol esters. As will be appreciated by one of skill in the art, other suitable mutants or combinations of mutants that are defective or deficient in sterol acylation may be used instead of are1are2.

In another aspect of the invention, there is provided a method of identifying sterol acyltransferase genes comprising:

(a) transforming a yeast mutant deficient in sterol ester synthesis with a nucleic acid molecule comprising a plant cDNA suspected of encoding a sterol acyltransferase; and

(b) detecting sterol ester formation in said yeast, wherein presence of increased sterol ester levels compared to a control indicates that said plant cDNA encodes a functional sterol acyltransferase.

As will be appreciated by one of skill in the art, the control may be an untransformed, mock transformed or vector transformed control and the control does not necessarily need to be repeated each time.

In those embodiments wherein the mutant is defective in sterol acylation, for example, are1are2, it is noted that simply the presence of sterol esters indicates that said plant cDNA encodes a functional sterol acyltransferase and no control is necessary.

In another embodiment, nucleotides that are at least 50%, 60%, 70%, 80%, 81%, 82%, 83%, 84%, 85%, 86%, 87%, 88%, 89%, 90%, 91%, 92%, 93%, 94% or 95% identical over their entire length to the polynucleotide encoding sterol acyltransferase of instant invention, that is, to SEQ ID No. 1 or SEQ ID No. 2, and polynucleotides that are complementary to such polynucleotides are disclosed.

In other embodiments, polynucleotides that encode polypeptides having substantially the same function as the sterol acyltransferase disclosed herein are disclosed. Conservative substitutions are specifically included.

In yet other embodiments, purified or isolated plant sterol acyltransferases comprising an amino acid sequence that is at least 60%, 65%, 70%, 75%, 76%, 77%, 78%, 79%, 80%, 81%, 82%, 83%, 84%, 85%, 86%, 87%, 88%, 89%, 90% or 95% homologous to SEQ ID No. 3 are disclosed. As will be appreciated by one of skill in the art, ‘isolated’ refers to polypeptides that have been ‘isolated’ from their native environment, in this case, from a plant cell, while ‘purified’ does not necessarily refer to absolute purity but rather refers to at least a 2, 3, 4 or 5 fold increase in purity. It is further of note that methods for identification of such plant sterol acyltransferase are described herein.

In yet a further embodiment, polynucleotides that hybridize to above disclosed sequences are disclosed. The hybridization conditions may be stringent in that hybridization will occur if there is at least a 90%, 95% or 97% identity with the polynucleotide that encodes the sterol acyltransferase of the instant invention. The stringent conditions may include those used for known Southern hybridizations such as, for example, incubation overnight at 42° C. in a solution having 50% formamide, 5×SSC (150 mM NaCl, 15 mM trisodium citrate), 50 mM sodium phosphate (pH 7.6), 5×Denhardt's solution, 10% dextran sulfate, and 20 micrograms/milliliter denatured, sheared salmon sperm DNA, following by washing the hybridization support in 0.1×SSC at about 65° C. Other known hybridization conditions are well known and are described in Sambrook et al., Molecular Cloning: A Laboratory Manual, Third Edition, Cold Spring Harbor, N.Y. (2001), incorporated herein in its entirety by this reference. In another embodiment, the present invention is directed towards homologs of the sterol acyltransferase gene described herein obtained from other organisms such as, for example, plants. Such homologs of the sterol acyltransferase gene described herein may be obtained by screening appropriate libraries that include the homologs, wherein the screening is performed with the nucleotide sequence of the plant sterol acyltransferase gene of the instant invention or portions or probes thereof.

In yet an additional embodiment, the nucleotide sequence of the sterol acyltransferase gene (FIG. 2, SEQ ID No. 1), the coding region (FIG. 3, SEQ ID No. 2), or the predicted amino acid sequence (FIG. 4, SEQ ID No. 3) of the instant invention may be used to search for homologous sequences using computer programs designed to search for homologous sequences. For instance, readily commercially available computer programs that may be used for such searches include without limitation, BLASTN, BLASTX and TBLASTX which may be used to search for nucleotide sequences, and BLASTP and TBLASTN which may be used to search for amino acid sequences. Such computer programs are readily accessible at the web-site www.ncbi.nlm.nih.gov.

In yet a further embodiment, a nucleotide sequence that codes for a sterol acyltransferase is transformed into a plant. As known in the art, there are a number of ways by which genes and gene constructs can be introduced into plants, and a combination of plant transformation and tissue culture techniques have been successfully integrated into effective strategies for creating transgenic crop plants. These methods, which can be used in the present invention, have been described elsewhere (Potrykus, 1991; Vasil, 1994; Walden and Wingender, 1995; Songstad et al., 1995), and are well known to persons skilled in the art. For example, one skilled in the art will certainly be aware that, in addition to Agrobacterium-mediated transformation of Arabidopsis by vacuum infiltration (Bechtold et al., 1993) or wound inoculation (Katavic et al., 1994), it is equally possible to transform other plant and crop species, using Agrobacterium Ti-plasmid-mediated transformation (e.g., hypocotyl (DeBlock et al., 1989) or cotyledonary petiole (Moloney et al., 1989) wound infection), particle bombardment/biolistic methods (Sanford et al., 1987; Nehra. et al., 1994; Becker et al., 1994) or polyethylene glycol-assisted, protoplast transformation (Rhodes et al., 1988; Shimamoto et al., 1989) methods.

As will also be apparent to persons skilled in the art, and as described elsewhere (Meyer, 1995; Dada et al., 1997), it is possible to utilize plant promoters to direct any intended up- or down-regulation of transgene expression using constitutive promoters (e.g., those based on CaMV35S), or by using promoters which can target gene expression to particular cells, tissues (e.g., napin promoter for expression of transgenes in developing seed cotyledons), organs (e.g., roots), to a particular developmental stage, or in response to a particular external stimulus (e.g., heat shock).

Plants that may be modified or used for sterol ester production according to the instant invention include, without limitation, borage (Borago spp.), Canola, castor (Ricinus communis); cocoa bean (Theobroma cacao), corn (Zea mays), cotton (Gossypium spp), Crambe spp., Cuphea spp., flax (Linum spp.), Lesquerella and Limnanthes spp., Linola, nasturtium (Tropaeolum spp.), Oeanothera spp., olive (Olea spp.), palm (Elaeis spp.), peanut (Arachis spp.), rapeseed, safflower (Carthamus spp.), soybean (Glycine and Soja spp.), sunflower (Helianthus spp.), tobacco (Nicotiana spp.), Vernonia spp., wheat (Triticum spp.), barley (Hordeum spp.), rice (Oryza spp.), oat (Avena spp.) sorghum (Sorghum spp.), rye (Secale spp.) or other members of the Gramineae. It will further be apparent by those of ordinary skill in the art that genomic or sequence libraries of each of these plants may be screened with the nucleotide or amino acid sequences of the instant invention for other sequences that encode or are homologous to sequences associated with the plant sterol acyltransferase of the instant invention.

As will be appreciated by one of skill in the art, the level of sterol-esters in plants are typically about 0.5% of total oil in seeds. As such, a transgenic plant comprising a non-native sterol acyltransferase gene as discussed herein will produce seeds having above 0.5% sterol-esters.

In an additional embodiment, knock-out mutants of plans are constructed. The plants are constructed by knocking-out the nucleotide sequence in the genome of the plants that codes for a sterol acyltransferase that is homologous to the sterol acyltransferase gene of the instant invention using known techniques.

In another embodiment; plants transformed with a nucleotide sequence of the instant invention that codes for a sterol acyltransferase and the knock-out mutants are studied for the impact of expressing the transformed nucleotide sequence that codes for the sterol acyltransferase or the lack of expression of the nucleotide sequence that codes for the sterol acyltransferase in knock-out mutants.

In yet a further embodiment, plants transformed with a nucleotide sequence of the instant invention that codes for a sterol acyltransferase are grown. Seeds of the transgenic plants are harvested and sterol-esters of the seeds are extracted. The extracted sterol esters are used for subsequent incorporation into a pharmaceutical composition, a nutraceutical composition or a food composition.

The invention will now be further explained and illustrated by way of examples; however, it is to be understood that the examples do not necessarily limit the invention and are primarily for illustrative purposes.

### Example I

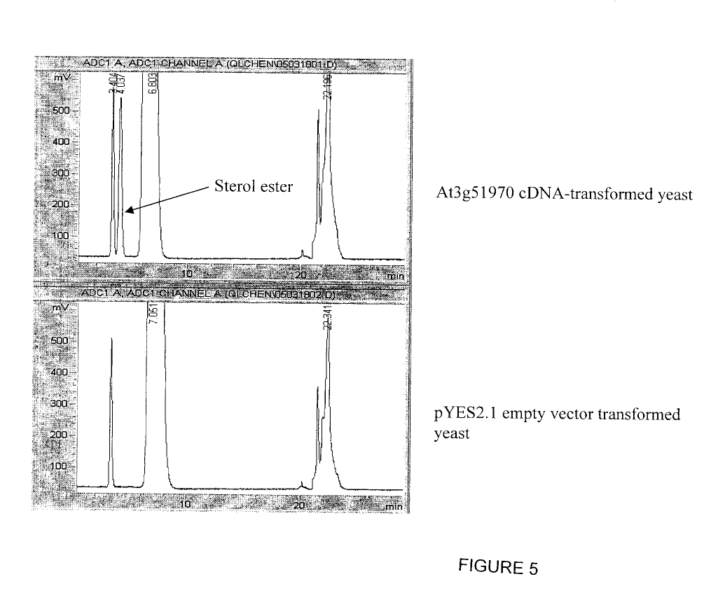

The Arabidopsis cDNA corresponding to the gene encoding a sterol acyltransferase, i.e., At3g51970 (FIG. 2), was cloned by RT-PCR using the commercial kit of SuperScript First-Strand Synthesis System for RT-PCR, commercially available from Invitrogen. The CDNA was sequenced and inserted into the vector pYES2.1N5-His-TOPO® (FIG. 1), commercially available from Invitrogen and used according to the manufacture's protocol. The vector having the inserted At3g51970 cDNA was transformed into the yeast are1/are2 mutant using the established procedure of Small-Scale Yeast Transformation as described in the pYES2.1N5-His-TOPO® manual from Invitrogen. Neutral lipid extracts of the yeast were subjected to normal-phase HPLC analysis to assay for the production of sterol esters in the yeast. The top panel of FIG. 5 illustrates the production of sterol esters in the yeast transformed with At3g51970, while the vector lacking At3g51970, i.e., pYES2.1 served as a negative control which lacked the sterol esters as illustrated in the bottom panel of FIG. 5.

The Arabidopsis gene, At3g51970, is a novel plant sterol acyltransferase gene and the full-length coding sequence is shown in FIG. 3. The amino acid sequence of At3g51970 is shown in FIG. 4.

### Example II Identification of a Brassica Sterol Acyltransferase Gene

The nucleotide and deduced, amino acid sequence information from Arabidopsis is used to search against Brassica genomic, cDNA and/or Expressed Sequence Tag information to identify a sterol acyltransferase gene from other Brassica species, i.e., Brassica napus. In another embodiment, the gene and/or the cDNA of At3g51970 is us as a labeled probe to carry out nucleotide hybridization to identify genes encoding sterol acyltransferase. In yet another embodiment, the polypeptide that is produced or generated according to sequence information of At3g51970 is used to generate antibody that is used to s for cDNA library for a sterol acyltransferase cDNA.

### Example III

Transformation of a plant with plant sterol acyltransferase gene.

The transformation protocol is adapted from that described by Bechtold et. al., (1993). Plants of Arabidopsis thaliana ecotype Columbia are grown in moist soil at a density of 10-12 plants per pot, in 4-inch square pots, and are covered with a nylon screen fixed in place with an elastic band. When the plants reach the stage at which bolts emerge, plants are watered, the bolts and some of the leaves are clipped, and the plants are infiltrated in Agrobacterium suspension as outlined below.

Agrobacterium transformed with the sterol acyltransferase gene of the instant invention is grown in a 25 mL suspension in LB medium containing kanamycin at a concentration of 50 μg/mL. The Agrobacterium is cultured for two to three days. The day before infiltration, this “seed culture” is added to 400 mL of LB medium containing 50 μglmL kanamycin. When the absorbance at 600 nm is >2.0, the cells are harvested by centrifugation (5,000 times g, 10 min in a GSA rotor at room temperature) and are re-suspended in 3 volumes of infiltration medium (1/.times Murashige and Skoog salts, 1 times. B5 vitamins, 5.0% sucrose, 0.044 μM benzylaminopurine) to an optical density at 600 nm of 0.8. The Agrobacterium suspension is poured into a beaker and the potted plants are inverted into the beaker so that the bolts and entire rosettes are submerged. The beaker is placed into a large Bell jar and a vacuum is drawn using a vacuum pump, until bubbles form on the leaf and stem surfaces and the solution starts to bubble a bit, and the vacuum is rapidly released. The necessary time and pressure vanes from one lab setup to the next; but good infiltration is visibly apparent as uniformly darkened, water-soaked tissue. Pots are removed from the beaker, are laid on their side in a plastic tray and are covered with a plastic dome, to maintain humidity. The following day, the plants are uncovered, set upright and are allowed to grow for approximately four weeks in a growth chamber under continuous light conditions as described by Katavic et al., (1995). When the siliques are mature and dry, seeds are harvested and selected for positive transformants.

### Example IV Selection of Putative Transformants (Transgenic Plants) and Growth and Analysis of Transgenic Plants

Seeds are harvested from vacuum-infiltration transformation procedures, and are sterilized by treating for 1 min in ethanol and 5 min in. 50% bleach/0.05% Tween 20™ in sterile distilled water. The seeds are rinsed several times with sterile distilled water. Seeds are plated by re-suspending them in sterile 0.1% agarose at room temperature (about 1 mL agarose for every 500-1000 seeds), and applying a volume equivalent to about 2,000-4,000 seeds onto 150.times.15 mm selection plates (½.times. Murashige and Skoog salts, 0.8% agar, autoclave, cool and add 1.times.B5 vitamins and kanamycin at a final concentration of 50 μg/mL). The plates are dried in a laminar flow hood until seed no longer flows when the plates are tipped. The plates are vernalized for two nights at 4° C. in the dark, and are moved to a growth chamber (conditions as described by Katavic et al., 1995). After 7-10 days, transformants are clearly identifiable as dark green plants with healthy green secondary leaves and roots that extend over and into the selective medium.

Seedlings are transplanted to soil, plants are grown to maturity and mature seeds (T₂ generation as defined in Katavic et al., 1994) are collected and analyzed. T₂ seeds are propagated. The vegetative growth patterns are monitored by measuring shoot tissue dry weights, and/or by counting the number of rosette leaves present by the time plants began to enter the generative (flower initiation) stage. Floral initiation (beginning of generative phase of growth) is analyzed by recording, on a daily basis, the percentage of plants in which a flower bud first appears and/or the percentage of plants that are bolting (as described by Zhang et al. 1997). Data is reported in terms of percentage of plants flowering/bolting on a given day after planting (d.a.p.).

### Example V

### Analysis of Sterol Esters

Cells or plants transformed with the sterol acyltransferase gene of the instant invention are grown to maturity and mature seeds are harvested. Neutral lipids are extracted from the cells or plants transformed with the sterol acyltransferase gene. The neutral lipids are subjected to normal-phase HPLC analysis to assay for the production of sterol esters in the transformed cells or plants.

While the preferred embodiments of the invention have been described above, it will be recognized and understood that various modifications may be made therein, and the appended claims are intended to cover all such modifications which may fall within the spirit and scope of the invention.

### REFERENCES

(The Contents of the Entirety of which. Each are Incorporated by this Reference)
Bechtold, N., Ellis, J. and Pellefer, G. (1993) In planta Agrobacterium-mediated gene transfer by infiltration of adult Arabidopsis thaliana plants. C.R. Acad. Sci. Ser. III Sci. Vie, 316: 1194-1199.
Becker, D., Brettschneider, R. and Lorz, H. (1994) Fertile transgenic wheat from microprojectile bombardment of scutellar tissue. Plant J. 5: 299-307.
Datla, R, Anderson, J. W. and Selvaraj, G. (1997) Plant promoters for transgene expression. Biotechnology Annual Review 3: 269-296.
DeBlock, M., DeBrouwer, D. and Tenning, P. (1989) Transformation of Brassica napus and Brassica oleracea using Agrobacterium tumefaciens and the expression of the bar and neo genes in the transgenic plants. Plant Physiol. 91: 694-701.
Katavic, Y., Haughn, G. W., Reed, D., Martin, M. and Kunst, L. (1994) In planta transformation of Arabidopsis thaliana. Mol. Gen. Genet. 245: 363-370.
Meyer, P. (1995) Understanding and controlling transgene expression. Trends in Biotechnology: 13: 332-337.
Moloney, M. M., Walker, J. M. and Sharma, K. K. (1989) High efficiency transformation of Brassica napus using Agrobacterium vectors. Plant Cell Rep. 8: 238-242.
Nehra, N. S., Chibbar, R. N., Leung, N., Caswell, K., Mallard, C., Steinhauer, L. Baga, M. and Kartha, K. K. (1994) Self-fertile transgenic wheat plants regenerated from isolated, scutellar tissues following microprojectile bombardment with two distinct gene constructs. Plant J. 5: 285-297.
Potrykus, L. (1991) Gene transfer to plants: Assessment of publish approaches and results. Annu. Rev. Plant Physiol. Plant Mol. BioL 42: 205-225.
Rhodes, C. A., Pierce, D. A., Mettler, I. J., Mascarenhas, D. and Detmer, J. J. (1988) Genetically transformed maize plants from protoplasts. Science 240: 204-207.

Sanford, J. C., Klein, T. M., Wolf, E. D. and Allen, N. (1987) Delivery of substances into cells and tissues using a particle bombardment process. J. Part. Sci. Technol. 5: 27-37.
Shimamoto, K., Terada, R., Izawa, T. and Fujimoto, H. (1989) Fertile transgenic rice plants regenerated from transformed protoplasts. Nature 335: 274-276.
Vasil, I. K. (1994) Molecular improvement of cereals. Plant Mol. Biol. 5: 925-937.
Walden, R. and Wingender, R. (1995) Gene-transfer and plant regeneration techniques. Trends in Biotechnology 13: 324-331.
Songstad D. D., Somers, D. A. and Griesbach, R. J. (1995) Advances in alternative DNA delivery techniques. Plant Cell, Tissue and Organ Culture 40: 1-15.
Zhan, H. Goodman, H. M. and Jansson, S. (1997) Antisense inhibition of photosystem I antenna protein Lhca4 in Arabidopsis thaliana. Plant Physiol. 115:1525-1531.

1-7. (canceled)

8. A process for increasing sterol ester production in a cell, the process comprising:
transforming a cell with a nucleic acid molecule encoding a sterol acyltransferase having an amino acid sequence that has at least 70% sequence identity to the amino acid sequence of SEQ ID NO: 3; and
growing the cell under conditions wherein said sterol acyltransferase is expressed.

9. (canceled)

10. The process of claim 8, wherein the nucleic acid molecule comprises a nucleotide sequence that is has at least 70% sequence identity to SEQ ID NO: 1 or 2.

11. The process of claim 8, further comprising isolating or purifying sterol esters from the cell.

12. The process of claim 11, further comprising admixing the isolated or purified sterol esters with a food composition or a pharmaceutically acceptable carrier.

13. The process of claim 11, further comprising admixing the isolated or purified sterol esters with a cholesterol lowering agent selected from the group consisting of plant sterols, psyllium, beta glucan, niacin, guggul extract, red rice yeast extract, policosanol, garlic, fenugreek, rice bran oil, fish oil, flaxseed oil, borage oil other omega-3-fatty acid containing oils, and combinations of any thereof.

14. The process of claim 8, wherein the cell is a bacterial cell, a yeast cell or a plant cell.

15. The process of claim 8, further comprising isolating the sterol acyltransferase.

16-17. (canceled)

18. A method of identifying sterol acyltransferase genes comprising:
(a) transforming a yeast mutant deficient in sterol ester synthesis with a nucleic acid molecule comprising a plant cDNA suspected of encoding a sterol acyltransferase; and
(b) detecting sterol ester formation in said yeast, wherein presence of sterol esters indicates that said plant cDNA encodes a functional sterol acyltransferase.

19. The process of claim 8, wherein the amino acid sequence has at least 80% sequence identity to the amino acid sequence of SEQ ID NO: 3.

20. The process of claim 8, wherein the amino acid sequence has at least 90% sequence identity to the amino acid sequence of SEQ ID NO: 3.

21. The process of claim 8, wherein the amino acid sequence has at least 95% sequence identity to the amino acid sequence of SEQ ID NO: 3.

22. The process of claim 8, wherein the amino acid sequence comprises SEQ ID NO: 3.

23. The process of claim 8, wherein the nucleic acid molecule comprises a nucleotide sequence that has at least 80% sequence identity to SEQ ID NO: 1 or 2.

24. The process of claim 8, wherein the nucleic acid molecule comprises a nucleotide sequence that has at least 90% sequence identity to SEQ ID NO: 1 or 2.

25. The process of claim 8, wherein the nucleic acid molecule comprises a nucleotide sequence that has at least 95% sequence identity to SEQ ID NO: 1 or 2.

26. The process of claim 8, wherein the nucleic acid molecule comprises SEQ ID NO: 1 or 2.
